(19) United States
(12) Patent Application Publication
(10) Pub. No.: US 2023/0320971 A1
(43) Pub. Date: Oct. 12, 2023

(54) SHAVING AID COMPOSITION

(71) Applicant: Edgewell Personal Care Brands, LLC, Chesterfield, MO (US)

(72) Inventor: Dana Vadnais, Stratford, CT (US)

(21) Appl. No.: 18/041,908

(22) PCT Filed: Sep. 2, 2021
(86) PCT No.: PCT/US2021/048871

§ 371 Date: Feb. 16, 2023

Related U.S. Application Data
(60) Provisional application No. 63/074,226, filed on Sep. 3, 2020.

(51) Int. Cl. A61K 8/73; A61K 8/86; A61K 8/02; A61K 8/60; A61Q 9/02

(52) U.S. Cl. CPC A61K 8/731; A61K 8/86; A61K 8/0216; A61K 8/608; A61Q 9/02; A61K 2800/31

(57) ABSTRACT

A shaving aid composition includes a water-soluble polymer, such as polyethylene oxide, and a cellulosic cationic polymer, such as a polyquaternium. The shaving aid composition may be substantially anhydrous and solid. A wet shave implement may include the shaving aid composition. For example, the shaving aid composition may be in a shaving aid strip, a lubrication box, or any combination thereof. A method to make a shaving aid may include selecting the water-soluble polymer, selecting the cationic polymer, and combining the water-soluble polymer and the cationic polymer to form the shaving aid composition. A method to use the shaving aid composition may include contacting the shaving aid composition with a solvent to activate the water-soluble polymer and to deposit at least the cellulosic cationic polymer on hair and/or skin from the wet shave implement during a shave.

### CROSS-REFERENCE TO RELATED APPLICATIONS

This application claims priority to International Application No. PCT/US2021/048871, filed Sep. 2, 2021, which claims priority to U.S. Provisional Patent Application No. 63/074,226, filed on Sep. 3, 2020, the content of which is incorporated herein for reference in its entirety.

### TECHNICAL FIELD

Embodiments relate to a shaving aid composition including a water-soluble polymer, such as polyethylene oxide, and a cellulosic cationic polymer, such as a polyquaternium.

### BACKGROUND

Water-soluble shaving aid compositions may be hydrolyzed and deposited during a shave to provide shave comfort. There is, however, considerable room for improvement to provide a shaving aid composition that enhances shave performance relative to commercialized shaving aid compositions.

### SUMMARY

Embodiments relate to a shaving aid composition including a water-soluble polymer and a cellulosic cationic polymer, wherein the cellulosic cationic polymer includes a cellulosic backbone having a substituted quaternary ammonium cation.

The shaving aid composition may be substantially anhydrous and solid. The shaving aid composition may include water in an amount of about 5.0 wt. % or less, based on a total weight of the shaving aid composition, as a substantially anhydrous shaving aid composition. For example, the shaving aid composition may include water in an amount of about 1.0 wt. % or less, based on a total weight of the shaving aid composition. The shaving aid composition may also be a collection of atoms or molecules held together so, under constant dry conditions, they maintain a defined shape and/or size as a solid shaving aid composition.

In any of the preceding embodiments, the shaving aid composition may include one or more auxiliary ingredients. For example, the shaving aid composition may include active agents, cleaning agents, processing aids, or combinations thereof. Auxiliary ingredients may be present in an amount of about 0.01 wt. % to about 40 wt. %, based on the total weight of the shaving aid composition. For example, a total amount of auxiliary ingredients in the shaving aid composition may be present in an amount of about 1.0 wt. % to about 20.0 wt. %, based on the total weight of the shaving aid composition.

In any of the preceding embodiments, a water-soluble polymer may include polyethylene oxide. When present, at least a portion of the polyethylene oxide may include one or more polyethylene oxide polymers having a molecular weight of about 100,000 Daltons to about 10,000,000 Daltons. For example, all PEO in the shaving aid composition may have a molecular weight of about 5,000,000 Daltons. In one example, the polyethylene oxide includes polyethylene glycol (PEG)-115M. For example, all PEO in the shaving aid composition may have a molecular weight of about 8,000,000 Daltons. In one example, the polyethylene oxide includes polyethylene glycol PEG-180M.

In any of the preceding embodiments, the water-soluble polymer may be present in an amount of 20 wt. % or more to 90 wt. % or less, based on the total weight of the shaving aid composition. For example, the water-soluble polymer may be present in an amount of about 40 wt. % to about 80 wt. %, based on the total weight of the shaving aid composition.

In any of the preceding embodiments, the cellulosic cationic polymer may have a % Nitrogen content greater than 1.1%. For example, the cellulosic cationic polymer may have a % Nitrogen content greater than 1.1% and less than 2.6%. In one example, the cellulosic cationic polymer may have a % Nitrogen content from 1.5% to 2.5%.

In any of the preceding embodiments, the cellulosic cationic polymer may have a viscosity less than 800 centipoise. For example, the cellulosic cationic polymer may have a viscosity greater than 100 centipoise and less than 800 centipoise. In one example, the cellulosic cationic polymer may have a viscosity from 300 centipoise to 500 centipoise.

In any of the preceding embodiments, the cellulosic cationic polymer may have a molar mass less than 1,000 kilograms/mole. For example, the cellulosic cationic polymer may have a molar mass greater than 100 kilograms/mole and less than 1,000 kilograms/mole. The cellulosic cationic polymer may have a molar mass, e.g., from 200 kg/mol to 800 kg/mol.

In any of the preceding embodiments, the cellulosic cationic polymer may be present in an amount of about 15 wt. % to about 60 wt. %, based on the total weight of the shaving aid composition. For example, the cellulosic cationic polymer may be present in an amount greater than 20 wt. % and less than 50 wt. %, based on the total weight of the shaving aid composition.

In any of the preceding embodiments, the cellulosic cationic polymer may include a polyquaternium. For example, the polyquaternium may include polyquaternium-10.

In any of the preceding embodiments, a ratio of the water-soluble polymer to the cellulosic cationic polymer may be from about 1:1 to about 5:1. For example, the ratio of the water-soluble polymer to the cellulosic cationic polymer may be from about 2:1 to about 4:1.

Embodiments further relate to a wet shave implement including the shaving aid composition in any of the preceding embodiments. In one example, the shaving aid composition may be disposed in a shaving aid strip, a lubrication box, or combinations thereof. Embodiments may, therefore, include coupling the shaving aid composition and the wet shave implement using known methods such as extrusion, compression molding, etc.

Embodiments further relate to a method to make the shaving aid composition in any of the preceding embodiments. For example, the method may include selecting the water-soluble polymer, selecting the cellulosic cationic polymer, and/or combining the water-soluble polymer and the cellulosic cationic polymer to form the shaving aid composition. Embodiments may, therefore, include combining (e.g., mixing, blending, etc.) ingredients of the shaving aid composition using known methods.

Embodiments further relate to a method to use the shaving aid composition in any of the preceding embodiments. For example, the method to use the shaving aid composition may include contacting the shaving aid composition with a solvent (e.g., water) to activate the water-soluble polymer (e.g., to cause polymer swelling) and to deposit at least the cellulosic cationic polymer on hair or skin from a wet shave implement during a shave.

### BRIEF DESCRIPTION OF THE DRAWINGS

The features of the disclosure believed to be novel and the elements characteristic of the invention are set forth with particularity in the appended claims. The disclosure itself, however, both as to organization and method of operation, can best be understood by reference to the description of the preferred embodiment(s) which follows, taken in conjunction with the accompanying drawings in which:

FIG. 1 is a graph showing differences in average scores for each of thirteen attributes (A1-A13) for an example shaving aid composition that is compared to a commercially available shaving aid control formulation according to embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. Any numerical range recited herein is intended to include all sub-ranges subsumed therein, and such ranges are understood to include each and every number and/or fraction between the stated range lower and upper values. Moreover, the term “about” and the like may refer to deviations of ±10% unless otherwise stated.

Wet Shave Implement

A wet shave implement may include a shaving aid strip. For example, shaving aid strips may be found in a commercialized product sold under the trade name SCHICK® (Edgewell Personal Care Brands, LLC). In one example, a shaving aid composition may be dispersed in a matrix of a water-insoluble material (e.g., a styrenic polymer, an olefinic polymer, etc.) to form a shaving aid composite that can be mounted in or on razor cartridge structures, adjacent a cutting edge or edges. Upon exposure to water, the shaving aid composition leaches from the matrix of the shaving aid strip and onto hair or skin. Thus, a shaving aid composition may be disposed in an erodible strip such as, without limitation, those described using the methods in commonly assigned United States Patent Application Publication No. 2017/0216442, which is incorporated herein in its entirety.

A wet shave implement may also include a lubrication box mounted on or adjacent to a razor cartridge. For example, lubrication boxes may be found in a commercialized product sold under the trade name SCHICK®. In one example, a shaving aid composition may be formed in-situ in an open body of the lubrication box or may be formed externally thereto and subsequently placed in the body of the lubrication box such that a plurality of apertures of the lubrication box allow the solubilized shaving aid composition to leach out of the lubrication box during a shave. Thus, a shaving aid composition may be included in a lubrication box such as, without limitation, those described using the methods in commonly assigned United States Patent Application Publication Nos. 2011/0099815, 2012/0023750, and 2018/0236677, which are incorporated herein in their entireties.

Shaving Aid Composition

A shaving aid composition may include any ingredient to enhance shave performance. For example, the shaving aid composition may include a water-soluble polymer such as, without limitation, polyethylene oxide (PEO), polyvinyl pyrrolidone, polyacrylamide, hydroxyethyl cellulose, hydroxypropyl cellulose, polyvinyl imidazoline, polyhydroxy ethylmethacrylate, poly vinyl alcohol, or combinations thereof. The water-soluble polymer may be present in an amount of 20 wt. % or more to 90 wt. % or less, preferably about 40 wt. % to about 80 wt. %, based on the total weight of the shaving aid composition.

Water-soluble polymers are readily commercially available. For example, PEO is commercially available as POLYOX™ or Foamysense™ from The Dow Chemical Company in a variety of molecular weights and particle sizes. As known in the industry and references in POLYOX™ or Foamysense™ commercial literature, the molecular weight of POLYOX™ or Foamysense™ polyethylene oxides, such as those, are determined largely by viscosity data. Thus, one or more PEOs may be selected based on a molecular weight (MW) of about 100,000 Daltons (Da) to about 10,000,000 Da, for example about 5,000,000 Da to about 8,000,000 Da. For example, PEO or a portion of PEO in a shaving aid composition may include higher molecular weight PEO (e.g., 2,000,000 Da or more), while in other embodiments all or a portion of PEO in the shaving aid composition may include lower molecular weight PEO (e.g., less than 2,000,000 Da). In one example, PEO may include a blend of about 40 wt. % to about 80 wt. % of total PEO having a MW of about 5,000,000 Da and about 20 wt. % to 60% of total PEO having a MW of about 300,000 Da. In another example, all of the water-soluble polymer consists of PEO and has a MW of about 5,000,000 Da. In a further example, all of the water-soluble polymer consists of PEO and has a MW of about 8,000,000 Da.

Preferably, PEO includes PEG-115M commercially available as POLYOX™ or Foamysense™ Coagulant, which has a MW of 5,000,000 Da, and which may be present in an amount of, for example, 36 wt. %, 50 wt. %, 88 wt. %, including every number and/or fraction therebetween, based on the total weight of the shaving aid composition. Additionally or alternatively, PEO may include PEG-180M commercially available as POLYOX™ or Foamysense™ 308, which has a MW of 8,000,000 Da, and which may be present in an amount of, for example, 36 wt. %, 50 wt. %, 88 wt. %, including every number and/or fraction therebetween, based on the total weight of the shaving aid composition.

The shaving aid composition may include a cellulosic cationic polymer. For example, the cellulosic cationic polymer may include a cellulosic backbone with a substituted quaternary ammoniur cation. The quaternary ammonium cation may be characterized with the general formula R₄N⁺ in the cationic form and with the general formula R₄N⁺X⁻ in the salt form, where (R₄) refers to the four group substituents of the nitrogen atom and (X⁻) refers to the counterion. The substituents of the quaternary ammonium cation may independently be, for example, substituted, unsubstituted, straight chain, branched chain, interrupted, and/or uninterrupted alkyl, aryl, alkylaryl, arylalkyl, cycloalkyl (aromatic or non-aromatic), heterocyclyl, and/or alkenyl groups. Moreover, two or more of the substituents Ra may together with the nitrogen atom form a substituted or unsubstituted heterocyclic ring. Interrupting groups may include heteroatoms such as oxygen, nitrogen, sulphur, and phosphorus-containing groups (e.g. phosphinate). Meanwhile, the most common counterion is chloride although other halides (e.g., fluoride, bromide, iodide, etc.) and non-halide ions (e.g., sulphonate, saccharine, etc.) may be used.

The cellulosic cationic polymer may be selected from one or more polyquaterniums. In one example, salts of hydroxyethyl cellulose may be reacted with trimethyl ammonium substituted epoxide to make polyquaternium-10, which is commercially available from, for example, Amerchol Corp., Edison, N.J., in the UCARE™ Polymer series of polymers. Polyquaternium-10 may generally be represented by the systematic name cellulose, 2-hydroxyethyl 2-[2-hydroxy-3-(trimethylammonio)propoxy]ethyl 2-hydroxy-3-(trimethylammonio)propyl ether, chloride (chloride salt form) (CAS No: 81859-24-7). Meanwhile, the UCARE™ polymers may be prepared by substituting trimethylammonium compounds onto hydroxyethyl cellulose, e.g., as shown by the following idealized structure:

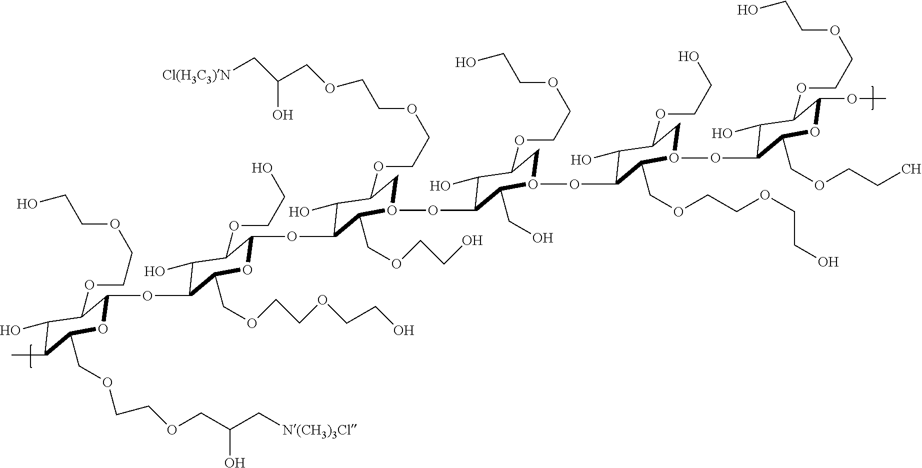

However, not all polyquaterniums may provide markedly enhanced shave performance in shaving aid compositions compared to commercialized shaving aid compositions. For example, it is known from zeta potential studies that the UCARE™ polymer JR-400 has less affinity for hair than the UCARE™ polymer JR-30M despite having substantially the same % Nitrogen content. Surprisingly however, shaving aid compositions unexpectedly achieve better shave performance with the UCARE™ polymer JR-400. Indeed, such shaving aid compositions surprisingly outperform others having different cellulosic cationic polymers with a higher % Nitrogen content and/or a higher viscosity than the polymer JR-400, discussed in further detail below. The UCARE™ polymer JR-400 may be commercially available in a mixture of >91.0 wt. % cationic polymer, 5.6 wt. % water, <1.5 wt. % sodium acetate, <1.5 wt. % sodium chloride, and 1.0 wt. % isopropanol, based on the total weight of the mixture.

Notably, the cellulosic cationic polymer may be selected based on a % Nitrogen content greater than 1.1%, for example 1.5% to 2.2%, and/or a viscosity less than 800 centipoise (Cps), for example 300 Cps to 500 Cps. The cellulosic cationic polymer may also have a molar mass less than 1,000 kilograms/mole (kg/mol), for example 200 kg/mol to 800 kg/mol. In addition, the cellulosic cationic polymer may be present in an amount of about 15 wt. % to about 60 wt. %, preferably greater than 20 wt. % and less than 50 wt. %, based on the total weight of the shaving aid composition. Thus, for example, the polymer JR-400 may be present in an amount of 20.1 wt. %, 25 wt. %, 30 wt. %, 49.9 wt. %, including every number and/or fraction therebetween, based on the total weight of the shaving aid composition.

The shaving aid composition may further include auxiliary ingredients such as, without limitation, cosmetically acceptable carriers, oils, sterols, amino acids, moisturizers, powders, colorants, pigments, dyes, pH adjusters, fragrances, cosmetic active ingredients, vitamins, preservatives, preservative boosters, foaming agents, chelating agents, cleansers, dispersing agents, thickeners, stabilizers, essential fatty acids, sphingolipids, emulsifiers, antioxidants, surfactants, additional film formers, chelating agents, gelling agents, thickeners, emollients, humectants, minerals, viscosity and/or rheology modifiers, keratolytics, retinoids, hormonal compounds, alpha-keto acids, anti-mycobacterial agents, anti-fungal agents, anti-microbials, anti-virals, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, stabilizers, pharmaceutical agents, spherical powders, extracts (fruit, flower, plant), absorbents, alpha and beta hydroxy acids, retinol and its derivatives, or combinations thereof.

For example, the shaving aid composition may include active agents, cleaning agents, processing aids, or combinations thereof. The active agents preferably include a therapeutic active agent including one or more of aloe, vitamins, humectants, emollients, moisturizers, clotting agents, anti-chafing agents, fragrances, depilatory agents, essential oils, antioxidants, alpha-hydroxy acids, alpha-keto acids, anti-bacterials, anti-fungals, anti-microbials, anti-virals, analgesics, anti-allergenics, antihistamines, anti-inflammatory agents, anti-irritants, anti-neoplastics, immune boosting agents, immune suppressing agents, anti-acne agents, anti-aging agents, anesthetics, antiseptics, insect repellents, skin cooling compounds, skin warming compounds, skin protectants, skin penetration enhancers, exfoliants, lubricants, or combinations thereof. The cleaning agents preferably include de-greasers, surfactants, enzymes, fabric conditioners, anti-microbials, anti-bacterials, anti-fungals, or combinations thereof. The processing aids preferably include emulsifiers, plasticizers, solubilizers, flexibilizers, rheology modifiers, fillers, or combinations thereof. In various embodiments, the processing aids include plasticizers such as polyethylene glycol, propylene glycol, butylene glycol, pentalene glycol, glycerin, or combinations thereof; flexibilizers comprising silicone resins, waxes, elastomers, polyamides, or combinations thereof; and/or fillers comprising boron nitride, graphite, talc, clay, silicates, diatomaceous earth, or combinations thereof.

Auxiliary ingredients may be present in an amount of about 0.01 wt. % to about 40 wt. %, based on the total weight of the shaving aid composition. An auxiliary ingredient may, for example, be present in an amount of 0.008 wt. %, 0.1 wt. %, 0.4 wt. %, 1.0 wt. %, 5.0 wt. %, 10.0 wt. %, 20.0 wt. %, 44.0 wt. %, including every number and/or fraction therebetween, based on the total weight of the shaving aid composition. For example, a total amount of auxiliary ingredients in the shaving aid composition may be present in an amount of about 1.0 wt. % to about 20.0 wt. %, based on the total weight of the shaving aid composition. In this regard, rice bran oil may be present in an amount of 0.01 to 0.5 such as, for example, 0.01, 0.05, 0.1, 0.2, 0.03, 0.4, 0.5, including every number and/or fraction therebetween, based on the total weight of the shaving aid composition.

The shaving aid composition may further include water in an amount of about 5.0 wt. % or less, based on a total weight of the shaving aid composition, as a substantially anhydrous shaving aid composition. In this regard, one or more ingredients of the shaving aid composition may have associated with it absorbed and/or adsorbed water, which can be carried into the otherwise substantially anhydrous shaving aid composition. For economic, sourcing, and/or formulation reasons, it may be disadvantageous to process hygroscopic materials to make them completely free of water. Thus, for example, the shaving aid composition in a wet shave implement may include about 1.0 wt. % or less total water.

Similarly, the shaving aid composition may be a collection of atoms or molecules held together so that, under constant dry conditions, they maintain a defined shape and size as a solid shaving aid composition. It is understood that in many end uses the shaving aid composition may be exposed to, and absorb, water as part of its designed function such as, for example, in a lubrication box that swells by exposure to water prior to contacting a user's hair or skin. Thus, the shaving aid composition in or on a wet shaving implement may be substantially anhydrous and solid before being solubilized prior to or during a shave.

### EXAMPLE(S)

Descriptive sensory analysis was conducted using panelists trained to detect and quantify sensations related to shave performance using shave implements having example shaving aid compositions listed in Table 1. This type of evaluation can be very useful in identifying and tracking specific changes among samples. Moreover, data collected is statistically robust. A variety of cellulosic cationic polymers, cationic guar polymers, cellulosic anionic polymers, and non-ionic polymers were also tested against control formulations that have only PEO, as shown in further detail in Table 2. However, only certain cationic cellulose polymers are discussed in detail below as representative of polymers that did not provide a significant different shave performance compared to the control formulations.

Referring now to Table 1, example shaving aid compositions include a water-soluble polymer. Example shaving aid compositions are formulated with polyethylene oxide selected from PEG-115M, PEG-180M, or combinations thereof. Thus, at least a portion of the polyethylene oxide includes one or more polyethylene oxide polymers having a MW of about 500,000 Daltons to about 8,000,000 Daltons. In addition, the cellulosic cationic polymer includes a cellulosic backbone having a substituted quaternary ammonium cation. Example shaving aid compositions are formulated with polyquaternium-10.

Example shaving aid compositions are manufactured by selecting the water-soluble polymer including polyethylene oxide, selecting the cellulosic cationic polymer including a cellulosic backbone having a substituted a quaternary ammonium cation, and combining the water-soluble polymer and the cellulosic cationic polymer using known methods. Example shaving aid compositions are substantially anhydrous and solid, and disposed in lubrication boxes for comparisons against similar commercially available products or other control.

TABLE I
Example Shaving Aid Compositions

| | | Approx. |
|---|---|---|
| Component (INCI) | Description | wt. % |
| Example Main Ingredients | | |
| PEG-115M (Foamysense ™ Coagulant), PEG- | Water-Soluble | 40-80 |
| 180M (Foamysense ™ 308) | Polymer | |
| Polyquaternium-10 (UCARE ™ JR-400) | Cellulosic | 20-50 |
| | Cationic Polymer | |
| Additional Ingredients | | |
| Sodium Chloride, Sodium Acetate, Zinc | Auxiliary | Q.S. 100 |
| Stearate, Isopropyl Alcohol, Ceramide NG, | Ingredients | |
| Cyclodextrin, Camellia Sinensis, Calendula | | |
| Officinalis, Oils (Hydrogenated Jojoba Oil, Rice | | |
| Bran), Panthenol, Sodium Hyaluronate, Aloe | | |
| (Leaf Juice), Tocopherol, Maltodextrin, and/or | | |
| Water (≤1 wt. %). | | |

Surprisingly, example shaving aid compositions unexpectedly achieved better shave performance with a combination of PEO (at a relatively lower amount) and the polymer JR-400 compared to control formulations including only PEO at relatively higher amounts. In the interest of brevity, the following results are discussed with reference to example shaving aid compositions formulated with PEG-115 (e.g., Example 1). It is noted, however, that additional example shaving aid compositions such as those formulated with PEG-180M, present in the same illustrative ratios and wt. %, exhibited similar sensory profiles as those discussed below.

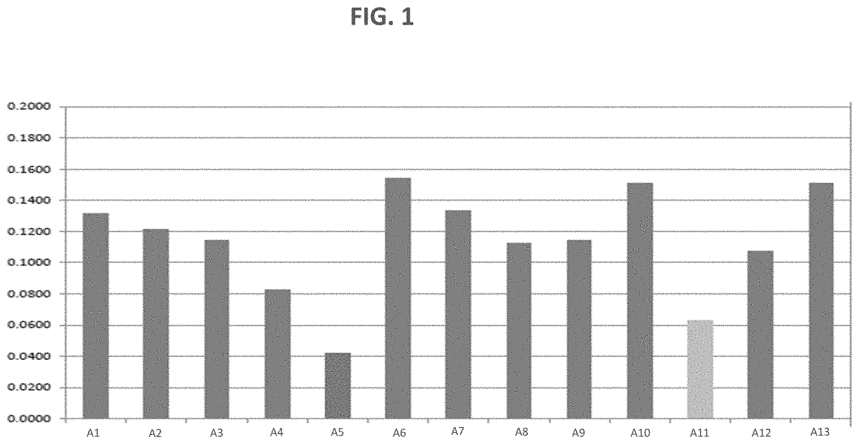

As shown by differences between the averages for each attribute (A1-A13) in FIG. 1, a shaving aid composition in a lubrication box having a ratio of PEO:JR-400 of about 3:1 (Example 1) scored statistically significantly better in twelve of the thirteen attributes tested in a two-week, five shave per each week test against a commercially available shaving aid composition (Comparative Example 1) as follows: overall liking (A1, 99% LOC); shaving comfort (A2, 99% LOC); ease of achieving a close shave (A3, 99% LOC); safe from nicks and cuts (A4, 95% LOC); lubricating quality JAR (A5, 90% LOC), glided easily over skin (A6, 99% LOC); did not pull or tug (A7, 99% LOC); left skin feeling smooth (A8, 99% LOC); left no stubble behind (A9, 99% LOC); caused no redness or irritation (A10, 99% LOC); lubrication amount (A12, 95% LOC); overall performance (A13, 99% LOC). Attribute A11, allowed more glide, had no significant difference between Example 1 and Comparative Example 1 evidencing that Example 1 still exhibited exceptional glide similar to that of Comparative Example 1.

Shaving aid compositions also unexpectedly achieved better shave performance with a combination of PEO and the polymer JR-400 than shaving aid compositions with other polyquaterniums when compared to the control formulations. Indeed, shaving aid compositions with the polymer JR-400 performed better than shaving aid compositions including polyquaterniums having a higher % Nitrogen content (e.g., 2.4-2.6) and/or a higher viscosity (e.g., 800 Cps-30,000 Cps) even when those polymers were present over a wide range of concentrations (e.g., 20 wt. %-55.15 wt. %).

Table 2 shows various properties, wt. %, and results for shaving aid compositions formulated similar to the example shaving aid compositions shown in Table 1, except that the polyquaternium-10 used was replaced by various other polymers and tested against control formulations having only PEO at relatively higher amounts.

TABLE II
Example Additional Comparative Shaving Aid Compositions

| Comparative | | Viscosity | | |
|---|---|---|---|---|
| Examples | % N | (Cps) | Wt. % | Improvement |
| 2 | 0.8-1.1 | 30,000 | 40 | N |
| | | (2% solution) | | |
| 3 | 1.5-2.2 | 30,000 | 30 | N |
| | | (2% solution) | | |
| 4-7 | 2.4-2.6 | 800-1800 | 20, 30, | N |
| | | (1% solution) | 40, 54.15 | |

Despite relatively high amounts of polyquaternium-10, Table 2 shows that the UCARE™ polymers LR-30M (Comparative Example 2) and JR-30M (Comparative Example 3) did not show significant improvement against control formulations having only PEO at relatively higher amounts. For example, Comparative Example 2 scored lower for stringiness (a good attribute linking to lubrication), lower for trimming (related to closeness attribute), lower for moustache soreness and oiliness, and higher for pulling/tugging compared to a control formulation during a two-day, full face test. Also, Comparative Example 3 scored lower in lubricity attributes (oiliness, stringiness) and higher for pulling/tugging against a control formula, which had less irritation during a two-week, split face test.

Additionally, 20 wt. % of Softcat™ 1300 SHX (Dow) (Comparative Example 4) did not show significant improvement in weeks 1 and 2 with advantage in lubricity towards a control formulation in weeks 3 and 4 during a four-week, split face test. Similarly, 30 wt. % of Softcat™ 1300 SHX (Comparative Example 5) was perceived to be less lubricious with lower stringiness, slipperiness, oiliness, and tackiness against a control formulation including only PEO at relatively higher amounts during a four-week, split face test. Also, 40 wt. % of Softcat™ 1300 SHX (Comparative Example 6) was perceived to be less lubricious than a control formulation during a four-week, split face test. Finally, 54.15 wt. % of Softcat™ 1300 SHX (Comparative Example 7) scored lower in almost all lubricity related attributes (stringiness, slipperiness, oiliness) against a control formulation including only PEO at relatively higher amounts that had higher glide, less redness on the throat, and less soreness on the throat and chin during a five-day, split face test.

Accordingly, a cellulosic cationic polymer may preferably be selected based on a % Nitrogen content greater than 1.1%, for example 1.5% to 2.2%, and/or a viscosity less than 800 Cps, for example 300 Cps to 500 Cps (2% solution). Moreover, a cellulosic cationic polymer may preferably have a molar mass less than 1,000 kg/mol, for example 200 kg/mol to 800 kg/mol. Additionally, a ratio of water-soluble polymer:cellulosic cationic polymer may preferably be from about 1:1 to about 5:1, more preferably from about 2:1 to about 4:1. Such ratios may be readily determined based on, for example, PEO used (e.g., in an amount of about 40 wt. % to about 80 wt. %, including any number and/or fraction therebetween), based on the total weight of the shaving aid composition, and polymer JR-400 used (e.g., in an amount greater than 20 wt. % and less than 50 wt. %, including any number and/or fraction therebetween), based on the total weight of the shaving aid composition, while allotting for a ±10% variance in concentration.

### ADDITIONAL NOTES AND EXAMPLES

Additional Example 1 may include a shaving aid composition comprising a water-soluble polymer including polyethylene oxide and a cellulosic cationic polymer comprising a cellulosic backbone including a substituted quaternary ammonium cation, wherein the shaving aid composition is substantially anhydrous and solid.

Additional Example 2 may include the shaving aid composition of Additional Example 1, wherein at least a portion of the polyethylene oxide includes one or more polyethylene oxide polymers having a molecular weight of about 100,000 Daltons to about 10,000,000 Daltons.

Additional Example 3 may include the shaving aid composition of any of Additional Examples 1 to 2, wherein the water-soluble polymer is present in an amount of about 40 wt. % to about 80 wt. %, based on the total weight of the shaving aid composition.

Additional Example 4 may include the shaving aid composition of any of Additional Examples 1 to 3, wherein the polyethylene oxide is selected from PEG-115M, PEG-180M, or combinations thereof.

Additional Example 5 may include the shaving aid composition of any of Additional Examples 1 to 4, wherein the cellulosic cationic polymer has a % Nitrogen content greater than 1.1%.

Additional Example 6 may include the shaving aid composition of any of Additional Examples 1 to 5, wherein the cellulosic cationic polymer has a % Nitrogen content from 1.5% to 2.5%.

Additional Example 7 may include the shaving aid composition of any of Additional Examples 1 to 6, wherein the cellulosic cationic polymer has a viscosity less than 800 centipoise.

Additional Example 8 may include the shaving aid composition of any of Additional Examples 1 to 7, wherein the cellulosic cationic polymer has a viscosity from 300 centipoise to 500 centipoise.

Additional Example 9 may include the shaving aid composition of any of Additional Examples 1 to 8, wherein the cellulosic cationic polymer is present in an amount greater than 20 wt. % and less than 50 wt. %, based on the total weight of the shaving aid composition.

Additional Example 10 may include the shaving aid composition of any of Additional Examples 1 to 9, wherein the cationic polymer includes polyquaternium-10.

Additional Example 11 may include the shaving aid composition of any of Additional Examples 1 to 10, wherein a ratio of the water-soluble polymer to the cellulosic cationic polymer is from about 2:1 to about 4:1.

Additional Example 12 may include a wet shave implement comprising the shaving aid composition of any of Additional Examples 1 to 11.

Additional Example 13 may include the wet shave implement of Additional Example 12, wherein the shaving aid composition is disposed in a shaving aid strip, a lubrication box, or combinations thereof.

Additional Example 14 may include a method to make the wet shave implement of any of Additional Examples 12 to 13, comprising positioning the shaving aid composition in the shaving aid strip, in the lubrication box, or combinations thereof.

Additional Example 15 may include a method to make the wet shave implement of Additional Example 14, further including coupling the wet shave implement with the shaving aid strip, the lubrication box, or combinations thereof.

Additional Example 16 may include a method to make the shaving aid composition of any of Additional Examples 1 to 11.

Additional Example 17 may include the method to make the shaving aid composition of Additional Example 16, comprising selecting a water-soluble polymer including polyethylene oxide, selecting a cellulosic cationic polymer comprising a cellulosic backbone including a substituted a quaternary ammonium cation, and combining the water-soluble polymer and the cellulosic cationic polymer to form the shaving aid composition, wherein the shaving aid composition is substantially anhydrous and solid.

Additional Example 18 may include the method to make the shaving aid composition of any of Additional Examples 16 to 17, wherein one or more polyethylene oxide polymers are selected based on a molecular weight of about 100,000 Daltons to about 10,000,000 Daltons.

Additional Example 19 may include the method to make the shaving aid composition of any of Additional Examples 16 to 18, wherein the cellulosic cationic polymer is selected based on a % Nitrogen content greater than 1.1%.

Additional Example 20 may include the method to make the shaving aid composition of any of Additional Examples 16 to 19, wherein the cellulosic cationic polymer is selected based on a % Nitrogen content from 1.5% to 2.5%.

Additional Example 21 may include the method to make the shaving aid composition of any of Additional Examples 16 to 20, wherein the cellulosic cationic polymer is selected based on a viscosity less than 800 centipoise.

Additional Example 22 may include the method to make the shaving aid composition of any of Additional Examples 16 to 21, wherein the cellulosic cationic polymer is selected based on a viscosity from 300 centipoise to 500 centipoise.

Additional Example 23 may include the method to make the shaving aid composition of any of Additional Examples 16 to 22, including combining the water-soluble polymer and the cellulosic cationic polymer to provide about 40 wt. % to about 80 wt. % of the water-soluble polymer and greater than 20 wt. % to less than 50 wt. % of the cellulosic cationic polymer, based on the total weight of the shaving aid composition.

Additional Example 24 may include the method to make the shaving aid composition of any of Additional Examples 16 to 23, including combining the water-soluble polymer and the cellulosic cationic polymer to provide a ratio in the shaving aid composition of the water-soluble polymer to the cationic polymer from about 2:1 to about 4:1.

Additional Example 25 may include the method to make the shaving aid composition of any of Additional Examples 16 to 24, wherein the polyethylene oxide is selected from PEG-115M, PEG-180M, or combinations thereof.

Additional Example 26 may include the method to make the shaving aid composition of any of Additional Examples 16 to 25, wherein the cationic polymer includes polyquaternium-10.

Additional Example 27 may include a method to use the shaving aid composition of any of Additional Examples 1 to 11, comprising contacting the shaving aid composition with a solvent to activate the water-soluble polymer and to deposit at least the cellulosic cationic polymer on a hirsute substrate or a keratinous substrate from a wet shave implement during a shave.

Additional Example 28 may include the method to use the shaving aid composition of Additional Example 27, comprising contacting the wet shave implement with the solvent, contacting the wet shave implement with a wet hirsute substrate, contacting the wet shave implement with a wet keratinous substrate, or combinations thereof.

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

What is claimed is:

1. A shaving aid composition comprising:
a water-soluble polymer comprising polyethylene oxide; and
a cellulosic cationic polymer comprising a cellulosic backbone including a substituted quaternary ammonium cation, wherein the shaving aid composition is substantially anhydrous and solid.

2. The shaving aid composition of claim 1, wherein at least a portion of the polyethylene oxide includes one or more polyethylene oxide polymers having a molecular weight of about 100,000 Daltons to about 10,000,000 Daltons.

3. The shaving aid composition of claim 1, wherein the water-soluble polymer is present in an amount of about 40 wt. % to about 80 wt. %, based on the total weight of the shaving aid composition.

4. The shaving aid composition of claim 1, wherein the polyethylene oxide is selected from polyethylene glycol (PEG)-115M, PEG-180M, or combinations thereof.

5. The shaving aid composition of claim 1, wherein the cellulosic cationic polymer has a % Nitrogen content greater than 1.1%.

6. The shaving aid composition of claim 5, wherein the cellulosic cationic polymer has a % Nitrogen content from 1.5% to 2.5%.

7. The shaving aid composition of claim 1, wherein the cellulosic cationic polymer has a viscosity less than 800 centipoise.

8. The shaving aid composition of claim 7, wherein the cellulosic cationic polymer has a viscosity from 300 centipoise to 500 centipoise.

9. The shaving aid composition of claim 1, wherein the cellulosic cationic polymer is present in an amount greater than 20 wt. % and less than 50 wt. %, based on the total weight of the shaving aid composition.

10. The shaving aid composition of claim 1, wherein the cationic polymer includes polyquaternium-10.

11. The shaving aid composition of claim 1, wherein a ratio of the water-soluble polymer to the cellulosic cationic polymer is from about 2:1 to about 4:1.

12. A wet shave implement comprising the shaving aid composition of claim 1.

13. The wet shave implement of claim 12, wherein the shaving aid composition is disposed in a shaving aid strip, a lubrication box, or combinations thereof.

14. A method to make a shaving aid composition comprising:
selecting a water-soluble polymer comprising polyethylene oxide;
selecting a cellulosic cationic polymer comprising a cellulosic backbone including a substituted a quaternary ammonium cation; and
combining the water-soluble polymer and the cellulosic cationic polymer to form the shaving aid composition, wherein the shaving aid composition is substantially anhydrous and solid.

15. The method of claim 14, wherein one or more polyethylene oxide polymers are selected based on a molecular weight of about 100,000 Daltons to about 10,000,000 Daltons.

16. The method of claim 14, wherein the cellulosic cationic polymer is selected based on a % Nitrogen content greater than 1.1%.

17. The method of claim 16, wherein the cellulosic cationic polymer is selected based on a % Nitrogen content from 1.5% to 2.5%.

18. The method of claim 14, wherein the cellulosic cationic polymer is selected based on a viscosity from 300 centipoise to 500 centipoise.

19. The method of claim 14, including combining the water-soluble polymer and the cellulosic cationic polymer to provide about 40 wt. % to about 80 wt. % of the water-soluble polymer and greater than 20 wt. % to less than 50 wt. % of the cellulosic cationic polymer, based on the total weight of the shaving aid composition.

20. The method of claim 14, including combining the water-soluble polymer and the cellulosic cationic polymer to provide a ratio in the shaving aid composition of the water-soluble polymer to the cationic polymer from about 2:1 to about 4:1.
